(19) United States
(12) Patent Application Publication
(10) Pub. No.: US 2020/0051660 A1
(43) Pub. Date: Feb. 13, 2020

(54) MODELING miRNA INDUCED SILENCING IN BREAST CANCER WITH PARADIGM

(71) Applicant: MANTOMICS, LLC, Culver City, CA (US)

(72) Inventors: Andrew SEDGEWICK, Culver City, CA (US); Shahrooz RABIZADEH, Culver City, CA (US); Patrick SOON-SHIONG, Culver City, CA (US); Charles Joseph VASKE, Culver City, CA (US)

(21) Appl. No.: 16/498,857

(22) PCT Filed: Mar. 27, 2018
(86) PCT No.: PCT/US2018/024615

§ 371 Date: Sep. 27, 2019

Related U.S. Application Data
(60) Provisional application No. 62/477,929, filed on Mar. 28, 2017.

(51) Int. Cl. G16B 5/20

(52) U.S. Cl. CPC G16B 5/20

(57) ABSTRACT

A probabilistic graphical pathway model is modified to include miRNA regulation by adding RISC as a regulatory factor. Most preferably, the pathway model is built using factor graphs, and the RISC includes DICER, TARBP2, and AGO2 or AGO1/3/4. So constructed pathway models can be used to infer RISC activity, which can be associated with various clinically relevant parameters to build various predictors or diagnostic tests.

This application claims priority to our copending US provisional application with the Ser. No. 62/477,929, which was filed Mar. 28, 2017.

### FIELD OF THE INVENTION

The field of the invention is computational pathway analysis using omics data to infer pathway activity and predict overall survival in cancer, especially where the pathway analysis uses miRNA data.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Current molecular taxonomy of breast cancer stems from classic mRNA expression profiling studies, which led to sub-classification of breast cancer into at least four distinct groups: luminal A, luminal B, basal-like, and HER2-positive. Further molecular analyses using miRNA expression in healthy breast tissue and breast cancer tissue to led to the identification of specific miRNAs that are indicative of the presence or progression, and even the sub-type of breast cancer. For example, an early study of miRNA expression in 86 breast tumors and normal tissues identified numerous miRNAs that were dysregulated in breast cancer, and allowed for the generation of a signature discriminating between normal and malignant breast tissues (see e.g., Cancer Res. 2005; 65, 7065-7070). Indeed, a signature of nine circulating miRNAs was also reported as capable of discriminating between early-stage breast cancer and healthy controls (Mol Oncol. 2014; 8:874-83).

Further detailed studies of breast cancer samples revealed specific miRNA signatures for luminal A and basal-type tumors, along with suspected functions of specific miRNAs in breast cancer (e.g., Molecular Oncology (2010) 230-241). Similarly, miRNA expression, mRNA expression, and DNA copy number were measured in human breast cancer in other studies, and based on the combined analysis of the miRNA and mRNA expression data, a number of miRNAs were identified that were differentially expressed between molecular tumor subtypes. In addition, a number of miRNAs were differentially expressed between the molecular tumor subtypes, and individual miRNAs were associated with clinicopathological factors. Indeed, selected miRNAs could classify basal versus luminal tumor subtypes in an independent data set (see e.g., Genome Biology 2007, 8:R214).

More recently, the potential use of miRNAs as prognostic and predictive biomarkers was investigated, and several miRNAs were described as putative therapeutic targets in breast cancer. For example, selected miRNAs were associated with hormone therapies, targeted therapies (e.g., ER, PR, HER2), and response to certain chemotherapeutic agents. Moreover, let-7b, miR-205, miR-375, miR-30a, miR-432-5p, and miR-497 were reported as being associated with positive prognosis (see Breast Cancer Research (2015) 17:21). However, despite the at least statistical association of certain miRNAs with breast cancer or a breast cancer sub-type, it remained unclear whether or not the siRNA would even be bound to the RNA-induced silencing complex (RISC). Likewise, the molecular composition of the RISC (e.g., AGO2, or AGO1/3/4) remained unclear, and with that an indication of the possible mechanism of action.

In still another study, RISC activity was investigated in hepatocellular carcinoma and investigators observed that higher RISC activity was associated with hepatocarcinogenesis. More specifically, the authors found that both AEG-1 (astrocyte elevated gene-1) and SND1 (staphylococcal nuclease domain containing 1, a RISC nuclease) were overexpressed, leading to increased activity of the RISC. However, it remained unclear which miRNA was/were bound to the RISC, and if increased RISC activity had any predictive power.

Therefore, even though numerous methods of miRNA analyses are known in the art, all or almost all of them suffer from various disadvantages. Consequently, there remains a need for improved systems and methods to use miRNA in omics analysis.

### SUMMARY OF THE INVENTION

The inventive subject matter is directed to systems and methods of analyzing omics data using miRNA, and especially omics analysis using a probabilistic graphical pathway model that use miRNA in addition to other omics data. In a preferred aspect of the inventive subject matter, the omics analysis is used to infer pathway activities for RISC in luminal A breast cancer to so predict overall survival.

In one aspect of the inventive subject matter, the inventors contemplate a method of quantifying RNA-induced silencing complex (RISC) activity in a tumor tissue of a patient that includes a step of obtaining omics data from a tumor tissue of the patient, wherein the tumor tissue is subtype luminal A breast cancer tissue, and that includes a further step of quantifying the RISC activity from the omics data using a probabilistic graphical pathway model having a plurality of pathway elements. Most typically, each of the pathway elements in the probabilistic graphical pathway model is represented by respective factor graphs, and at least one of the factor graphs models the RISC and comprises at least one of AGO1, AGO2, AGO3, and AGO4.

It is generally contemplated that the omics data typically comprise copy number data, transcription level data, and miRNA data, and it is further generally contemplated that the probabilistic graphical pathway model uses a priori known miRNA and respective miRNA targets. In some embodiments, the factor graph that models the RISC comprises AGO2. Where desired, contemplated methods may include a step of comparing the quantified RISC activity with a threshold level, and optionally a step of updating a patient record when the quantified RISC activity is above the threshold level or a step of associating a clinical parameter with the quantified RISC activity.

Therefore, and viewed from a different perspective, the inventors also contemplate a method of detecting RNA-induced silencing complex (RISC) activity in a tumor tissue of a patient. In such method, omics data are obtained from the tumor tissue of the patient, and the RISC activity is detected in the patient by inputting the omics into a probabilistic graphical pathway model, wherein the probabilistic graphical pathway model uses a priori known miRNA and respective miRNA targets. The RISC activity is then calculated from the omics data in the pathway model.

Most typically, the omics data comprise copy number data, transcription level data, and miRNA data, and the probabilistic graphical pathway model uses a plurality of factor graphs. Moreover, it is generally preferred that at least one of the plurality of factor graphs models RNA-induced silencing complex (RISC) comprising at least one of AGO1, AGO2, AGO3, and AGO4. While not limiting the inventive subject matter, it is also preferred that the probabilistic graphical pathway is PARADIGM. Contemplated methods may also include a step of comparing the quantified RISC activity with a threshold level, and optionally a step of updating a patient record when the quantified RISC activity is above the threshold level or associating a clinical parameter with the quantified RISC activity.

In further contemplated embodiments, a method of predicting overall survival of a patient having subtype luminal A breast cancer is contemplated, wherein the method includes a step of obtaining omics data from a tumor tissue of the patient. In a further step, the RISC activity is quantified from the omics data using a probabilistic graphical pathway model having a plurality of pathway elements, wherein each of the pathway elements in the probabilistic graphical pathway model is represented by respective factor graphs, and wherein at least one of the factor graphs models the RISC with AGO2. Finally, the patient is then diagnosed as having a decreased overall survival when decreased RISC-AGO2 activity is detected.

As noted above, it is contemplated that the omics data in such method comprises copy number data, transcription level data, and miRNA data, and/or that the probabilistic graphical pathway model (preferably PARADIGM) uses a priori known miRNA and respective miRNA targets.

Consequently, the inventors also contemplate a computer system for omics analysis that includes an omics database that is informationally coupled to an analysis engine, wherein the omics database stores omics data of a patient. Most preferably, the analysis engine is programmed to: receive the omics data from the omics database, and to calculate, using the omics data and a probabilistic graphical pathway model a RISC activity. Most typically, the probabilistic graphical pathway model has a plurality of pathway elements, and wherein each of the pathway elements in the probabilistic graphical pathway model is represented by respective factor graphs, and at least one of the factor graphs models the RISC and comprises at least one of AGO1, AGO2, AGO3, and AGO4.

In most embodiments, the omics data comprise copy number data, transcription level data, and miRNA data, and/or the probabilistic graphical pathway model (e.g., PARADIGM) uses a priori known miRNA and respective miRNA targets. It is also generally preferred that the factor graph that models the RISC comprises AGO2, or at least one of AGO1, AGO3, and AGO4. Preferably, but not necessarily, the analysis engine is also programmed to compare the quantified RISC activity with a threshold level. Additionally, the analysis engine may also be programmed to update a patient record when the quantified RISC activity is above the threshold level or to associate a clinical parameter with the quantified RISC activity.

Various objects, features, aspects and advantages of the inventive subject matter will become more apparent from the following detailed description of preferred embodiments, along with the accompanying drawing.

### BRIEF DESCRIPTION OF THE DRAWING

FIG. 1A is an exemplary depiction of a factor graph for a single pathway element.

FIG. 1B is an exemplary depiction of a RISC complex using logical connections following the example factor graph of FIG. 1A.

FIG. 1C depicts a detail view of a modified factor graph for the pathway element of FIG. 1A in which the pathway element is regulated by a RISC complex as illustrated in FIG. 1B.

FIG. 1D depicts a detail view of a modified factor graph for the pathway element of FIG. 1A in which the pathway element is regulated by an endoculeolytic RISC complex as a transcriptional regulator.

FIG. 1E depicts a detail view of a modified factor graph for the pathway element of FIG. 1A in which the pathway element is regulated by an non-endoculeolytic RISC complex as a transcriptional regulator.

FIG. 2 is an exemplary heat map of inferred pathway activities comparing miRNA, AGO1/3/4 RISC, and AGO2 RISC influence on pathways in various breast cancer subtypes.

FIG. 3A is an exemplary violin plot depicting inferred pathway activity distribution for AGO2 RISC grouped by breast cancer subtype.

FIG. 3B is an exemplary violin plot depicting inferred pathway activity distribution for AGO1/3/4 RISC grouped by breast cancer subtype.

FIG. 4 is a survival prediction plot for luminal A type breast cancer comparing high and low AGO2 RISC in patients.

### DETAILED DESCRIPTION

The inventors have discovered that by adding miRNAs, miRNA target predictions, and a model of the RNA induced silencing complex to a pathway analysis model (e.g., to PARADIGM), updated models could be created that can interrogate miRNA induced gene silencing in a pathway context. Based on a comparison between a transcription regulation only model to an updated RISC model that regulates genes at the transcriptional and/or translational level, the inventors noted that such updated models are significantly better able to learn miRNA-target links at the transcriptional regulation level.

For example, and based on recently developed pathway analysis systems and methods as described in more detail in WO 2011/139345, WO/2013/062505, and WO/2014/059036, incorporated by reference herein, the inventors now contemplate that pathway analysis and pathway model modifications of PARADIGM can be employed in silico to predict overall survival in breast cancer, and especially luminal A subtype breast cancer using DNA copy number data, RNA expression data, and miRNA data from breast cancer tissue samples.

PARADIGM is a pathway based algorithm that allows for the integration of multiple genomic data types with a curated pathway database to make pathway activity predictions. In the present inventive subject matter, the inventors added a model of miRNA-mediated gene silencing to the PARADIGM algorithm to study miRNA expression in a pathway context. More particularly, as is shown in more detail below, the inventors curated a set of 7751 miRNA-mRNA interactions from the union of three target prediction algorithms (TargetScan, PicTar, miRanda). These interactions involved 66 miRNA and 2814 mRNA transcripts. The so updated PARADIGM algorithm was run on copy number, RNAseq and miRNAseq data from 697 patients in the TCGA breast cancer cohort, and changes in the learned interactions between active miRNAs and their targets between different subtypes were investigated. As is shown in more detail below, the updated PARADIGM algorithm included RISC with (DICER1, TARBP2, AGO2+miRNA) and without (DICER1, TARBP2, AGO1/3/4+miRNA) endonucleolytic activity.

In some embodiments the miRNA-target pairs that exhibited the largest correlation changes between Basal and Luminal A breast cancer subtypes were enriched for known oncogenes, and for miRNAs and genes related to the activity of miRNAs in cancer. In addition, these targets were involved in a number of relevant signaling pathways including PI3K-AKT, JAK-STAT, RAP1 and RAS. Most of these highly differential links involved the miR-16 family of miRNAs which are known tumor suppressors. Two miRNA-mRNA target pairs showed the largest changes in link strength of any pathway links between Basal and Luminal A groups. The miRNAs in these pairs, miR-195 and miR-221, are both previously documented markers in breast cancer. Therefore, by looking at changes in miRNA-target links between tumor subtypes, the updated PARADIGM algorithm allowed identification of both miRNAs and target genes involved in pathways relevant to breast cancer.

As is well known, miRNA are short (18-25 nucleotide) non-coding RNA molecules that target mRNA transcripts and silence genes via a variety of mechanisms. Gene silencing due to miRNA targeting plays a part in many biological processes, and miRNA target sequences have been predicted in as many as 30% of genes. Dysregulation of miRNAs has been linked to a variety of human diseases, and miRNAs have been studied extensively in cancer as noted above. In order to post-transcriptionally silence genes, miRNAs associate with several proteins to form an RNA Induced Silencing Complex (RISC) that carries out the biological process that leads to silencing. While the molecules involved in the formation of RISC can vary considerably, the minimal component proteins in human are the RNAase Dicer, which processes the miRNA transcripts into a mature form, the Argonaute family of proteins, the catalytic component of RISC, and TRBP, which recruits the Argonaute proteins to Dicer and the bound miRNA molecule.

While the general structure of RISC is known, proper identification of the mRNA that is targeted by each miRNA remains a significant challenge. In fact, due to the difficulty of experimental verification of miRNA-mRNA targeting, there are only relatively few validated targets. Therefore, a variety of in silico methods were developed to predict targeting based on factors such as sequence, binding energy, and conservation. Unfortunately, common results among these methods are typically low relative to the number of predicted targets. Therefore, the inventors used the union of several of these in silico methods to identify a set of high-confidence target predictions. In the present example, the inventors used the set union of TargetScan, miRanda, and PicTar.

A number of previous studies have attempted to combine miRNA target predictions with either pathway data (PloS one 7: e42390), mRNA expression (Bioinformatics: btu489), or both (Nucleic Acids Res 39 (Web Server issue): W416-423). The MirSystem (PloS one 7: e42390) links miRNA to pathway knowledge via their mRNA targets, and then performs enrichment tests to determine which pathways are likely to be regulated by a given group of miRNAs. Zhang's system (Bioinformatics: btu489) uses causal learning methods combined with matched miRNA and mRNA data to predict miRNA activity in a condition specific manner. On the other hand, MirConnX (Nucleic Acids Res 39 (Web Server issue): W416-423) combines matched miRNA and mRNA data with target predictions and transcription factor regulation data to find condition specific regulatory networks.

In contrast, the updated PARADIGM algorithm offered several advantages over these known methods. First, while a number of these methods offer condition specific models, the updated PARADIGM algorithm is able to model patient-specific pathway activities, which allow for more flexible downstream analyses. In addition, the currently known methods study paired miRNA and mRNA data by looking at pairwise correlations between the miRNA-target pairs, while the updated PARADIGM algorithm enables investigation of the miRNA-target RNA interaction using predictions of active miRNA silencing complexes. Thus, if proteins essential to the silencing pathway such as Argonaute or DICER are not active in the sample, the updated PARADIGM algorithm will predict less miRNA regulation in that sample.

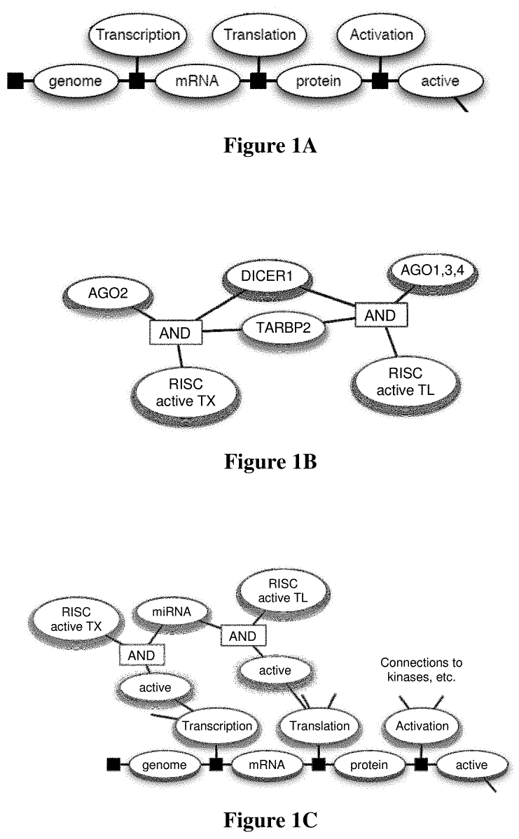

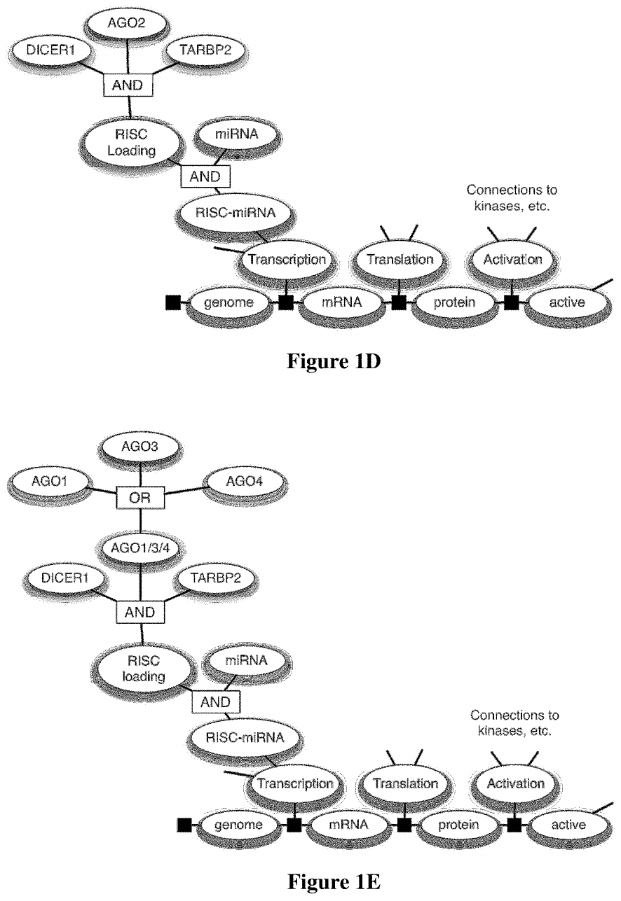

As noted above (see WO 2011/139345, WO/2013/062505, and WO/2014/059036), PARADIGM builds a factor graph from a curated database of pathways in order to infer unobserved levels of activity of individual proteins, protein complexes, and families from observed DNA and mRNA data. Observed data is discretized to three levels corresponding to high, low and normal. For every protein in the PARADIGM pathway, a model of the central dogma of molecular biology is included in the factor graph as shown in FIG. 1A. This central dogma means that each protein-coding gene in a cell will have identical central dogma structure, and it is therefore possible to share parameters between all genes. For example, a single pathway entity (e.g., receptor, kinase, transcription factor, etc.) may be expressed in a factor graph as shown in FIG. 1A where the entity is represented by its specific variables such as genome data, mRNA data, protein data, and activity data, which may be measured, or inferred. Viewed from a different perspective, it should be appreciated that each step in the dogma may have an unobserved variable in the graph: DNA, RNA, protein, and active (for activated protein or protein activity). Each of these latent variables is linked to observed data, if available, and to active variables of other genes that could be annotated as regulators in the pathway database. The states of the latent variables are then inferred from the data using loopy-belief propagation to perform Expectation-Maximization to so arrive at a probabilistic graphical pathway model as already described WO 2011/139345, WO/2013/062505, and WO/2014/059036.

A typical pathway model represents a digital model of activity of a target omic system to be modeled, preferably in the form of a factor graph. Each pathway model will therefore typically comprise a plurality of pathway elements, such as members of a signal transduction network (e.g., receptors, kinases, phophorylases, transcription factors, etc.). Between at least two pathway elements is at least one regulatory node. Thus, at least two pathway elements are coupled to each other via a path having a regulatory node, and the regulatory node controls activity along the path between the elements as a function of one or more regulatory parameters. One should appreciate that a pathway model can include any practical number of pathway elements, regulatory nodes, and regulatory parameters. Most typically, however, a pathway element will include a DNA sequence, an RNA sequence transcribed from the DNA sequence, a protein encoded in the RNA, and a protein function of the protein, or any other activity elements.

For example, where a pathway element comprises a DNA sequence, regulatory parameters can include a transcription factor, a transcription activator, a RNA polymerase subunit, a cis-regulatory element, a trans-regulatory element, an acetylated histone, a methylated histone, a repressor, or other activity parameters. Additionally, in scenarios where the pathway element comprises an RNA sequence, regulatory parameters can include an initiation factor, a translation factor, a RNA binding protein, a ribosomal protein, an siRNA, a polyA binding protein, or other RNA activity parameter. Still further, where the pathway element comprises a protein, regulatory parameters could include phosphorylation, an acylation, a proteolytic cleavage, or an association with at least a second protein. It should be appreciated that while relationships between the variables in PARADIGM are set, the parameters of the factors, which model the relationships between the nodes they connect, are learned by the algorithm. Thus, although it is not possible to learn new edges with PARADIGM, by looking at the regulation parameters learned from the observed data, one can measure how strong an edge is in a given set of samples.

In the modified PARADIGM algorithm contemplated herein, miRNA is included using the same dogma that protein coding RNAs use. The only dogma variable that does not apply to miRNA is the ‘protein’ variable (node), and since there are not translational or activation regulators for the miRNA in the pathway, the ‘active’ variable will have the same state as the ‘RNA’ variable for a miRNA with high probability. The present RISC model uses the built-in complex model in PARADIGM, which is a “noisy AND” function. In other words, the predicted activity state of the complex is the minimum of the states of all the components of the complex with high probability, or another state with small error probabilities. FIG. 1B is an exemplary illustration in which the RISC is modeled in a factor graph as shown in FIG. 1A, and which provides putative regulation mechanisms (TX, transcription control; TL, translation control) of the different proteins in the Argonaute family Argonaute 2 (AGO2) is part of a complex that regulates transcription because of its endoribonuclease activity that allows it to cleave mRNA molecules thereby silencing them. Although this process occurs post-transcription, kinetic studies of cleavage by AGO2 suggest that it occurs rapidly enough that it will affect observed mRNA transcript levels.

The rest of the Argonaute family (AGO1/3/4) was treated as translational regulators because their alternative silencing mechanisms are less likely to affect the observed mRNA transcript levels. These mechanisms include translation regulation activity such as direct translational repression via recruitment of additional factors and deadenylation of the poly(A) tail of the mRNA molecule, which in turn inhibits translation. Consequently, these different regulation models interact with the regulation nodes of a predicted target protein as shown in FIG. 1C. While the full model with both transcriptional and translational repression by RISC as presented in FIG. 1C can be used, a simpler model may be employed that only adds the transcriptional regulation component corresponding to mRNA cleavage by AGO2.

It should therefore be appreciated that by inference or actual measurements of various RISC components (e.g., copy number DNA, transcription level or measured quantities of miRNA, and/or protein quantity/activity), the factor graphs for a probabilistic graphical pathway model can be modified to now also include the (measured or inferred) effects of miRNA. Thus, it should be recognized that miRNA information can now be used beyond simple association of a miRNA with a disease or condition to appropriately predict and/or quantify physiological or genetic effects of the miRNA. Moreover, such effects may be differentiated by mechanism of action where the type of Argonaute protein is taken into consideration (e.g., AGO2 for endonucleolytic action on mRNA or AGO1/3/4 for inhibition of translation).

Of course, it should be noted that the pathway model modification as described above need not be limited to a modification of paradigm, but that all known pathway models can be modified by applying measured or inferred quantities of the RISC complex (and especially TARBP2, DICER, Argonaute proteins, and miRNA) to the pathway models in a manner that allows modification of transcription and/or translation activity of a gene that is subject to miRNA regulation. Therefore, and more generally, it should be appreciated that contemplated systems and methods are suitable to investigate the effects of RNA silencing, even if a particular association of a miRNA with a specific target is unknown. Indeed by using contemplated system and methods that integrate RISC activity into a (probabilistic) pathway model, clinical relevant features associated with increased or decreased RISC activity (RISC with AGO2 or AGO1/3/4) can be identified and subsequently used in a predictive or analytic model, typically using conventional machine learning algorithms Moreover, identification of clinical relevant features also provides further insight into targets or miRNA that would otherwise not be readily identified.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

### EXAMPLE

miRNA Target Predictions: The intersection of miRNA-mRNA target predictions from 3 miRNA target prediction algorithms was used: TargetScan (URL: targetscan.org), miRANDA (URL: microRNA.org), and PicTar (URL: pictar.mdc-berlin.de). The database of targets comes from mirConnX (URL: benoslab.pitt.edu/mirconnx/). This procedure generated 7751 miRNA-mRNA interactions involving 66 miRNA and 2814 mRNA.

Omics data: The inventors used matched RNAseq, miRNAseq and DNA copy number data for 697 patients from the TCGA Breast Cancer Cohort. For the DNA copy number data GISTIC 2.0 predictions were used. To normalize the RNAseq data, transcripts with zero reads were removed in more than 50% of samples, TPM values were log-scaled, and each transcript median normalized across all samples. For miRNA normalization filtered miRNAs with zeros reads were filtered in more than 75% of samples, then the raw counts were log scaled and each miRNA was median normalized across all samples. For validation of the PARADIGM model, the inventors uses Reverse Phase Protein Array (RPPA), hormone receptor status from immunohistochemistry, survival, and PAM50 subtype predictions for these patients.

PARADIGM modification: PARDAIGM (NantOmics, 9920 Jefferson Blvd. Culver City, Calif. 90232) was modified by incorporating into the factor graphs the RISC model as shown in FIG. 1B to arrive at an updated factor graph representation as is shown in FIG. 1C. AGO1, AGO3, and AGO4 were treated as a “family” node in the factor graph (inputs combined in a logical OR function). AGO2 and the AGO1/3/4 family combine with DICER1 and TARBP2 to form “complex” nodes in the factor graph (logical AND operation), representing RISC loading complexes with and without endonucleolytic activity. Every miRNA node in the pathway combines with these loading complexes form active silencing complexes that regulate the predicted activity of the mRNA targets of the loaded miRNA by attaching to the transcriptional regulation node of the mRNA pathway as an inhibitor.

In further modifications (used for data discussed below), the AGO2 complexes and the AGO1/3/4 complexes were attached as transcriptional only regulators, and an exemplary mode of attachment for these complexes is shown in FIGS. 1D and 1E.

Survival prediction: To see how well the Integrated Pathway Activities (IPAs) predicted by the different PARADIGM models represent the underlying biology of the tumors, the inventors studied how well they were able to predict patient survival. This task was treated as a classification problem where the two classes are patients in the top quartile or the bottom quartile of survivals. Due to incomplete survival data for many patients, the data set was limited to 30 patients, 15 high survival and 15 low survival. The miRNA transcription regulation model performed best, an SVM (support vector machine) trained on IPAs from this model achieved a leave-one-out cross validation accuracy of 60% while the full model achieved poor accuracy of 43%, as did a model learned without any miRNA data, 37% accuracy. The performance of the simpler model is comparable to doing the classification with RNAseq data (59% accuracy) or RNAseq and miRNAseq data together (62% accuracy).

Correlation of IPAs with IHC Data: Another method for validating our models is to compare to other data types. The inventors compared the IPAs from each model for ESR1 and the ER! homodimer to compare to estrogen receptor status as measured by IHC, and for ERBB2 to compare to IHC measured HER2 status. The IHC experiment gives a call of positive or negative for each hormone receptor, so a two sample ranksum test of the IPAs was performed for the positive versus negative groups of the corresponding hormone receptor. All three models performed well on these tests. The full miRNA model (regulated transcription and translation) had highly significant p-values from the tests: 2.9e-48 for ESR1, 2.9e-47 for the ER! homodimer, and 1.2e-9 for ERBB2. The transcription-only miRNA model has slightly lower p-values: 1.4e-49 for ESR1, 7.9e-48 for ER! homodimer, and 2.8e-11 for ERBB2. The original PARADIGM model without any miRNA data had the lowest p-values for ESR1 (8.5e-50) and ERBB2 (9.1e-12), but the highest for ER! homodimer (9e-46).

Top miRNA-target Links: Although the above noted tests did not clearly separate either of the miRNA regulation models, the inventors choose to focus on the links learned by the transcription-only model because it performed better in all tests and as the transcription regulation links are likely to have more informative parameters. In further studies, it was investigated how the miRNA-target links change between breast cancer subtypes, specifically when comparing 97 patients with aggressive basal tumors to 288 patients with more treatable luminal A tumors.

The inventors sorted miRNA-target links by the largest change in correlation between the basal and luminal A subgroups. Of the top 10 links with large correlation changes between the groups, 9 of them involve miR-16. This is likely due to the very low IPA of miR-16 in basal tumors (median—4.0) compared to luminal A tumors (median 0) (Wilcoxon p<2e-16). miR-16 is a known tumor suppressor that has been characterized in a variety of cancers including lymphoma, leukemia and breast cancer. The targets of the top 200 links by correlation change are significantly enriched (false discovery rate<0.05) for a number of pathways relevant to cancer, as shown in more detail below. Table 1 below shows KEGG enrichment for the gene targets of the top 200 miRNA-target links by correlation change between basal and luminal A breast cancer subgroups.

TABLE 1

| | Pathway | | |
|---|---|---|---|
| Pathway | Size | Number Found | FDR |
| Jak-STAT signaling pathway | 32 | 8 | 3.499e−06 |
| Rap1 signaling pathway | 70 | 10 | 1.506e−05 |
| PI3K-Akt signaling pathway | 95 | 9 | 2.470e−03 |
| MicroRNAs in cancer | 78 | 8 | 4.457e−03 |
| Melanoma | 23 | 5 | 4.649e−03 |
| Pathways in cancer | 132 | 10 | 5.570e−03 |
| Focal adhesion | 65 | 7 | 1.121e−02 |
| Regulation of actin cytoskeleton | 67 | 7 | 1.362e−02 |
| Endocytosis | 70 | 7 | 1.806e−02 |
| Ras signaling pathway | 73 | 7 | 2.360e−02 |
| HTLV-I infection | 73 | 7 | 2.360e−02 |
| Proteoglycans in cancer | 73 | 7 | 2.360e−02 |
| Wnt signaling pathway | 55 | 6 | 3.732e−02 |
| Transcriptional misregulation | 57 | 6 | 4.546e−02 |
| in cancer | | | |

In addition to correlation, the inventors also used a G-test to measure the statistical dependence of the variables, which we refer to as link “strength”. The G-test allows to uncover links that are highly dependent, but do not necessarily have a linear relationship that can be captured by Pearson's correlation. Looking at the rank difference of G-test p-values between the basal and luminal groups, it was observed that two miRNA regulation links had the largest change out of all links in the pathway: miR-221-ARF4 shows a strong connection in the luminal A subgroup (FDR=9.9e-7), but a relatively weaker relationship in basal tumors (FDR=1.5e-3). Both nodes in this link have been previously linked to breast cancer: overexpression of miR-221 is linked to aggressive, basal tumors through promotion of epithelial-to-mesenchymal transition, and ARF4 expression is linked to cell migration and metastasis in breast cancer. Similarly, miR-195-BDNF has a strong silencing relationship in luminal A tumors (FDR=5.6e-21) that is weaker in basal patients (FDR=3.2e-3). miR-195 has been identified as a potential circulating biomarker to diagnose breast cancer and BDNF is a growth factor that has been shown to promote tumor growth and proliferation in colon cancer.

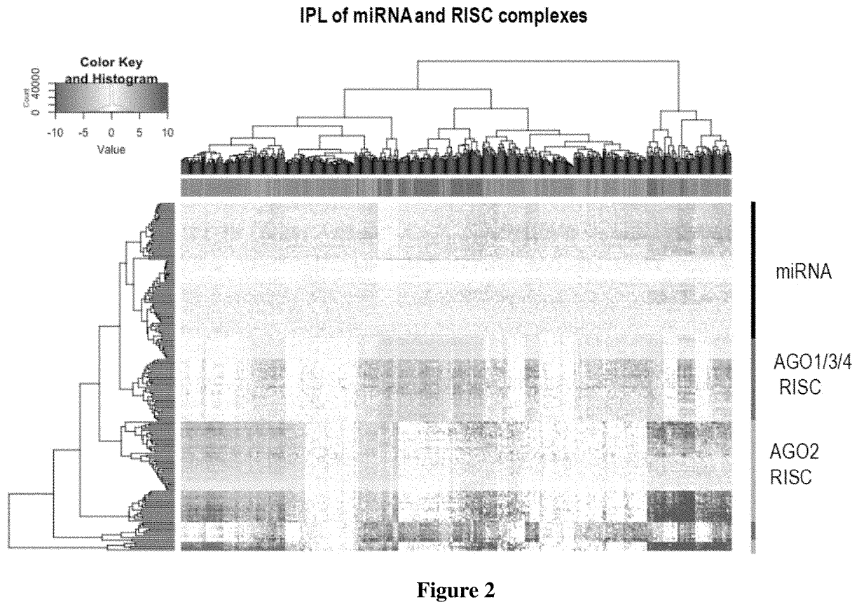

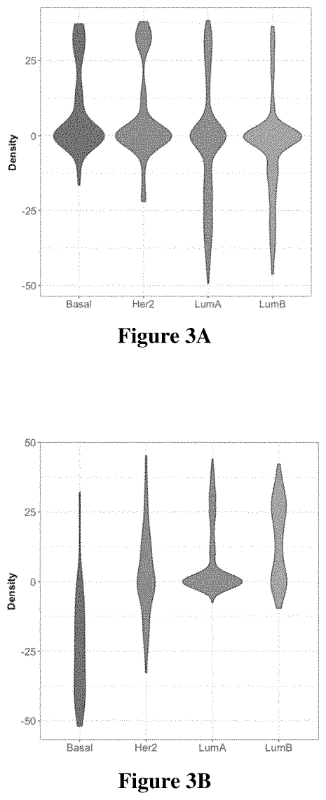

Using the probabilistic graphical pathway analysis model PARADIGM modified as discussed above on TCGA data, the inventors further investigated whether or not the particular type of RISC complex (endonucleolytic, with AGO2 protein, or non-endonucleolytic, with AGO1, 3, or 4 protein) would have an influence of inferred pathway activity (also: integrated pathway likelihood). As can be readily seen from FIG. 2, inferred pathway activities for miRNA only do not exhibit significant differences across all patients. Also, no specific grouping is evident for the four distinct subtypes (having the same color coding as in FIGS. 3A and 3B). Only moderate consistent differences in the inferred pathway activities can be seen where RISC includes any one of AGO1, AGO3, and AGO4. However, some decreased activity seems to correlate with basal type (see also FIG. 3B). The most significant differences were observed where RISC included AGO2 and where the subtype was luminal A.

FIGS. 3A and 3B illustrate these findings in violin plots where the cancer subtypes are individually plotted against the change in activity. More specifically, FIG. 3A shows inferred pathway activities (IPL) with respect to RISC with AGO2 for each of the subtypes of breast cancer. Here, higher pathway activities have higher positive values, and the thickness of the plot is representative of the number of patients with a given level of pathway activity. As is readily apparent, basal and Her2 type cancers have mostly upregulated inferred pathway activities for RISC with AGO2, while Luminal A and Luminal B have upregulated inferred pathway activities for RISC with AGO2. On the other hand, as depicted in FIG. 3B, basal breast cancer has mostly downregulated pathway activities for RISC with AGO1/3/4, while Luminal A and Luminal B have upregulated inferred pathway activities for RISC with AGO1/3/4. The Her2 subtype had up- and downregulated inferred pathway activities for RISC with AGO1/3/4.

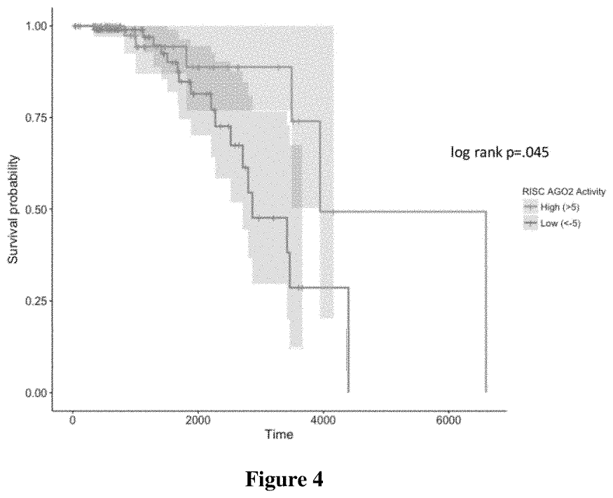

Based on these observations, the inventors then set out to determine whether or not these differences in RISC activity would correlate with survival time (or any other patient specific parameter such as drug resistance, likely treatment outcome using immune therapy, etc.). To that end, inferred RISC activities were associated with overall survival data from the TCGA database and supervised machine learning using SVM package was performed on the data, where activity was set as ‘high’ for inferred pathway activation of >5 and as ‘low’ for inferred pathway activation of <5 (see also FIG. 3A), and FIG. 4 shows exemplary results for that analysis. As can be seen from the graph, high RISC with AGO2 activities were statistically correlated with increased survival time. Therefore, it should be appreciated that pathway analysis using RISC/miRNA influence can be used to predict various clinical parameters associated with RNA silencing, where the parameters may be cancer specific, stage specific, and/or subtype specific.

Furthermore, contemplated methods also allowed for the identification of regulatory link changes and associated targets, thus providing additional information that may identify new or confirm known targets of RNA silencing using specific miRNA. Table 2 exemplarily lists the largest inferred pathway activity (IPL) differences in high vs. low Luminal A patients and Table 3 exemplarily lists regulatory link changes between Luminal A and Basal patients.

TABLE 2

| | | Median |
|---|---|---|
| | | IPL |
| Entity | Rank Sum FDR | Difference |
| RISC_AG02_(complex) | 5.2e−11 | 51.11 |
| MIR9_(family)_RJSC.AG02_(complex) | 5.2c-11 | 22.68 |
| MIR1_(family)_RISC- | 5.2e−11 | 21.25 |
| 1_G02_(complex) | | |
| MIR195_RISC_AG02_(complex) | 5.2e−11 | 19.70 |
| MIR186_RISC_AG02_(complex) | 5.2e−11 | 18.28 |
| NFATC2 | 1.9e−20 | −3.95 |
| SP1 | 1.8e−23 | −4.20 |
| MYB | 2.0e−23 | −6.00 |
| MYC/Max_(complex) | 6.2e−20 | −6.03 |
| p53_tetramer_(complex) | 6.3e−10 | −10.98 |

TABLE 3

| miRNA | Target | Basal Correlation | Luminal A Correlation |
|---|---|---|---|
| MIR113_RISC_AGO2_(complex) | TXNIP | −0.59 | −0.07 |
| MIR107_RISC_AGO2_(complex) | RAB1B | −0.42 | 0.13 |
| MIR141_RISC_AGO2_(complex) | TTR | −0.86 | −0.31 |
| MIR7_(family)_RISC-AG01/3/4- | C20orf24 | −0.32 | 0.28 |
| (complex) | | | |
| MIH93_RISC_AGO2_(complex) | C7orf43 | −0.33 | 0.32 |
| MIR7_(family)_RISC- | FOXF2 | 0.29 | −0.77 |
| AG01/3/4_(complex) | | | |
| MIR7_(family)_RISC- | POGFRI3 | 0.22 | −0.79 |
| AG01/3/4_(complex) | | | |
| MIR24_(family)_RISC- | LRRC32 | 0.24 | −0.74 |
| AG01/3/4_(complex) | | | |
| MIR24_(family)_RISC- | PDGFRA | 0.24 | −0.73 |
| AG01/3/4_(complex) | | | |
| MIR211_RISC-AG01/3/4 | ANGPTL2 | 0.18 | −0.76 |

In view of the above, it should therefore be appreciated that the mechanisms of RNA silencing are different between the Luminal A subtype and the Basal type, which thus may indicate different treatment modalities that may be chosen. In addition, the inventors could demonstrate that low endonucleolytic RISC activity (especially low RISC with AGO2) was strongly associated with more aggressive tumors. Therefore, contemplated system and methods are particularly useful in the prediction of overall survival in Luminal A type breast cancer, and with that provide a tool to recommend a more aggressive treatment strategy.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of quantifying RNA-induced silencing complex (RISC) activity in a tumor tissue of a patient, comprising:
obtaining omics data from a tumor tissue of the patient, wherein the tumor tissue is subtype luminal A breast cancer tissue;
quantifying the RISC activity from the omics data using a probabilistic graphical pathway model having a plurality of pathway elements;
wherein each of the pathway elements in the probabilistic graphical pathway model is represented by respective factor graphs; and
wherein at least one of the factor graphs models the RISC and comprises at least one of AGO1, AGO2, AGO3, and AGO4.

2. The method of claim 1 wherein the omics data comprise copy number data, transcription level data, and miRNA data.

3. The method of claim 1 wherein the probabilistic graphical pathway model uses a priori known miRNA and respective miRNA targets.

4. The method of claim 1 wherein the factor graph that models the RISC comprises AGO2.

5. The method of claim 1 further comprising comparing the quantified RISC activity with a threshold level.

6. The method of claim 5 further comprising updating a patient record when the quantified RISC activity is above the threshold level or associating a clinical parameter with the quantified RISC activity.

7. A method of detecting RNA-induced silencing complex (RISC) activity in a tumor tissue of a patient, comprising:
obtaining omics data from the tumor tissue of the patient; and
detecting the RISC activity in the patient by inputting the omics into a probabilistic graphical pathway model, wherein the probabilistic graphical pathway model uses a priori known miRNA and respective miRNA targets; and
calculating from the omics data in the pathway model a RISC activity.

8. The method of claim 7 wherein the omics data comprise copy number data, transcription level data, and miRNA data.

9. The method of claim 7 wherein the probabilistic graphical pathway model uses a plurality of factor graphs.

10. The method of claim 9 wherein at least one of the plurality of factor graphs models RNA-induced silencing complex (RISC) comprising at least one of AGO1, AGO2, AGO3, and AGO4.

11. The method of claim 7 wherein the probabilistic graphical pathway is PARADIGM.

12. The method of claim 7 further comprising comparing the quantified RISC activity with a threshold level.

13. The method of claim 12 further comprising updating a patient record when the quantified RISC activity is above the threshold level or associating a clinical parameter with the quantified RISC activity.

14. A method of predicting overall survival of a patient having subtype luminal A breast cancer, comprising:
obtaining omics data from a tumor tissue of the patient;
quantifying the RISC activity from the omics data using a probabilistic graphical pathway model having a plurality of pathway elements;
wherein each of the pathway elements in the probabilistic graphical pathway model is represented by respective factor graphs, and wherein at least one of the factor graphs models the RISC with AGO2; and
diagnosing the patient as having a decreased overall survival when decreased RISC-AGO2 activity is detected.

15. The method of claim 14 wherein the omics data comprise copy number data, transcription level data, and miRNA data.

16. The method of claim 14 wherein the probabilistic graphical pathway model uses a priori known miRNA and respective miRNA targets.

17. The method of claim 14 wherein the probabilistic graphical pathway is PARADIGM.

18. A computer system for omics analysis, comprising:
an omics database informationally coupled to an analysis engine;
wherein the omics database stores omics data of a patient;
wherein the analysis engine is programmed to:
(a) receive the omics data from the omics database;
(b) calculate, using the omics data and a probabilistic graphical pathway model a RISC activity;
(c) wherein the probabilistic graphical pathway model has a plurality of pathway elements, and wherein each of the pathway elements in the probabilistic graphical pathway model is represented by respective factor graphs; and
(d) wherein at least one of the factor graphs models the RISC and comprises at least one of AGO1, AGO2, AGO3, and AGO4.

19. The computer system of claim 18 wherein the omics data comprise copy number data, transcription level data, and miRNA data.

20. The computer system of claim 18 wherein the probabilistic graphical pathway model uses a priori known miRNA and respective miRNA targets.

21. The computer system of claim 18 wherein the probabilistic graphical pathway is PARADIGM.

22. The computer system of claim 18 wherein the factor graph that models the RISC comprises AGO2.

23. The computer system of claim 18 wherein the factor graph that models the RISC comprises at least one of AGO1, AGO3, and AGO4.

24. The computer system of claim 18 wherein the analysis engine is further programmed to compare the quantified RISC activity with a threshold level.

25. The computer system of claim 24 wherein the analysis engine is further programmed to update a patient record when the quantified RISC activity is above the threshold level or to associate a clinical parameter with the quantified RISC activity.
